(19) United States
(12) Patent Application Publication
(10) Pub. No.: US 2018/0110932 A1
(43) Pub. Date: Apr. 26, 2018

(54) Cap Needle Assembly for an Injection Device with Predetermined Breaking Point

(71) Applicant: Novo Nordisk A/S, Bagsvaerd (DK)

(72) Inventor: Jacqueline Trosborg, Kongens Lyngby (DK)

(21) Appl. No.: 15/568,518

(22) PCT Filed: Apr. 21, 2016
(86) PCT No.: PCT/EP2016/058865

§ 371 Date: Oct. 23, 2017

(30) Foreign Application Priority Data
Apr. 29, 2015 (EP) 15165686.5

(51) Int. Cl. A61M 5/32; A61M 5/34

(52) U.S. Cl. CPC A61M 5/3202; A61M 2005/312; A61M 5/347

(57) ABSTRACT

A needle assembly (1, 100) for an injection device comprising a needle cannula (10, 10), a hub (30, 130) comprising skirt portion (40, 140) and a central portion (60, 60). The skirt portion (40, 140) is having an internal surface (43, 143) adapted for connection to an injection device, wherein the skirt portion (40, 140) is having an open proximal end (41, 141) adapted to be sealed off by a sealing member (90, 190). The hub (30, 130) is having a distal end (42, 142) closed by the central portion (60, 160), wherein the central portion (60, 160) is adapted to receive the cannula (10, 110). The internal surface (43, 143) of the skirt portion (40, 140) comprises a connecting member, a cap portion (80, 180) for covering the distal end (12, 112) of the cannula (10, 110), a frangible zone (89, 189). The cap portion (80, 180) can be turned from an initial mode, wherein the frangible zone (89, 189) connects the cannula cap portion (80, 180) and the hub (30, 130), to a broken mode, wherein the frangible zone (89, 189) is broken, and wherein the cannula cap portion (80, 180) and the hub (30, 130) can be separated from each other. The sealing member (90, 190) is permeable to a sterilizing agent, and thereby adapted to enable sterilization of an internal space (5, 105).

### TECHNICAL FIELD

The invention relates to a needle assembly for an injection device having a removable cap. The invention also relates to a method of using and producing a needle assembly.

### BACKGROUND

Needle assemblies for injection devices are commonly used to inject substances into human or animal bodies. Such needle assemblies are typically disposable and are discarded after only one use.

Traditionally a needle unit for an injection device comprises a hub, a needle cannula and a removable inner cap is provided in a secondary packaging comprising an outer container and a removable seal. Thereby, a needle assembly comprising both a needle unit and secondary packaging typically comprises at least 5 parts. Such a needle assembly can for example be seen in US 2012/0029440 A1.

In order to shield a needle from damage, to protect the medical professionals from inadvertent contact, and to preserve sterility of a needle prior to use needles are typically provided with a removable cap. Examples of such caps can be seen in US 2012/0029440 A1 U.S. Pat. No. 6,053,892, and U.S. Pat. No. 8,323,251 B2. The needle cap may also optionally provide assurance to the end user that the needle has not been tampered with, as shown in U.S. Pat. No. 6,053,892 and U.S. Pat. No. 8,323,251 B2. Furthermore, U.S. Pat. No. 8,323,251 B2 allegedly discloses a needle assembly pre-connected with an injection device, and that the needle cap can be configured to operate a needle from an initial position to an actuated position, where the needle has pierced a sealing disk. The assembly is adapted to be pre-assembled by press-fitting the assembly onto the injection device, in an axial motion, and the cap operates the needle from the initial to the actuated position by a turning movement.

Each part of the needle assembly is both costly in production and assembly, and increases the number of use steps for the patient. For example for the needle assembly shown in US 2012/0029440 A1, the user would have to remove both an outer container, and an inner cap before use.

GB 927, 626 and U.S. Pat. No. 3,073,307 discloses a disposable needle hub and a cannula sheath structure adapted to be mounted on the outlet end of a hypodermic syringe. The hub comprises a hypodermic needle hub section of a non-vitreous material adapted to be mounted on a hypodermic syringe, and a severable but integral needle cannula sheath section sealably closed at its outer end and integrally joined at the inner end thereof with the hub section to sealably enclose one end of a needle cannula mountable therein.

U.S. Pat. No. 2,371,086 discloses a hypodermic injector which is convenient to use, and which provides a correct dossage that readily can be injected into a patient. The document further discloses a collapsible container for the dosage and a hypodermic needle communicating with or adapted to communicate with the container, and in which the needle or the upper part thereof is contained within a frangible seal.

DE 847473 describes needle assemblies comprising a cap portion with a frangible zone.

WO 20/102067 describes a needle assembly comprising an outer container 44. De 4140101 describes a needle assembly comprising a protective cover for a blood-sample needle. The protective cover comprises an outer sheath and a collar. The outer sheath is joined across a predetermined break point to the collar which carries the needle. Inside the outer sheath, at least in the region of the point of the needle, is an inner sheath surrounding the needle. The inner sheath and the outer sheath are manufactured by two-component injection moulding. The inner sheath is secured to the outer sheath by form-locking and/or force-locking connections so that it cannot be displaced along the needle.

Having regard to the above, it is an object of the present invention to provide a cost-effective needle assembly, with a minimum of components, attractive handling properties for the user, and wherein the needle unit can be disposed after use without disposing the injection device.

It is a further object of the invention to provide a needle assembly adapted to be easily provided in a sterile condition.

It is further an object of the invention to provide a medical system comprising such a needle unit and a corresponding injection device. It is further an object of the present invention to provide a method of mounting and producing such a needle assembly.

### SUMMARY

In the disclosure of the present invention, embodiments and aspects will be described, which will address one or more of the above objects or which will address objects apparent from the below disclosure as well as from the description of exemplary embodiments.

In a first aspect, a needle assembly for an injection device is provided, wherein the needle assembly comprises a needle cannula and a hub. The hub comprises a skirt portion and a central portion, wherein the skirt portion is having an internal surface adapted for connection to an injection device. The skirt portion is having an open proximal end adapted to be sealed off by a sealing member, and the hub is having a distal end closed by or connected to the central portion. The central portion is adapted to receive the cannula, and the internal surface of the skirt comprises a connecting member, wherein the connecting member is adapted to be turnably connectable with a corresponding connecting member on an injection device. The needle assembly further comprises a sealing member, a cap portion for covering the distal end of the cannula, and a frangible zone. The cap portion can be manipulated or turned from an initial mode, to a broken mode. In the initial mode the frangible zone connects the cannula cap portion and the hub, and in the broken mode, the frangible zone is broken, and the cannula cap portion and the hub can be separated from each other. The needle assembly comprises a sterilizable mode, wherein the cannula cap portion, the hub, the frangible zone and the sealing member, comprises an outer surface surrounding an internal space of the needle assembly. The sealing member is permeable to a sterilizing agent, and is thereby adapted to enable sterilization of the internal space. The outer surface is adapted to preserve sterility of the internal space.

The sterilizing agent can be a sterilizing gas, a sterilizing vapour or a sterilizing hot steam.

The needle hub serves as a part of the outer surface, and is thereby a part of the outer protection against microorganisms. It is not necessary to envelope the assembly in a sterile bag, or outer container which is typically seen for medical equipment. In a condition, where the needle assembly is sterile, sterility can be preserved by the cap portion, the hub and the sealing member. In a condition, where the needle assembly is not sterile, the needle assembly can, due to the permeable sealing barrier, be sterilized in a bulk process.

A sterilizable mode is defined as a mode where the cap portion, the hub and the sealing member are connected to each other, and where a sterilizing agent can enter through the sealing barrier. The internal of the assembly may be sterile or unsterile, when the assembly is in the sterilizable mode.

In one aspect of the invention, the connecting member can be adapted to be reversibly or releasably connected to the injection device during mounting. After use, the needle assembly can be disconnected from the injection device and be disposed. In the broken mode, the cap portion and the hub can be separated from each other in an axial motion, i.e., the cap portion can be pulled off.

Also or alternatively, in an aspect of the invention, the skirt portion further comprises a proximal sealing surface adapted to attach to the sealing member, and the sealing member is adapted to attach to the sealing surface for covering the proximal end of the hub.

Hereby is provided a self-contained needle assembly, which is easy to sterilize and to preserve in a sterile condition. The needle assembly is self-contained and is thereby a unit which can be handled and stored separate from the injection device.

Also or alternatively, the attached sealing member is adapted to be peeled off. Hereby is provided a needle assembly, wherein the sealing between the hub and the sealing member can be inspected, i.e., has the peeling started, as it is easy to see or inspect whether the assembly is preserved in a sterile condition or not.

Also or alternatively, the proximal sealing member may be adapted to be broken or pierced, when the needle is mounted on an injection device.

Hereby is provided a needle assembly, wherein the sealing between the hub and the sealing member can be inspected, i.e., is the sealing member broken, as it is easy to see if the assembly is preserved in a sterile condition.

Also, in some embodiments the cap portion, the frangible zone and the hub are moulded in one piece. To provide a cost-effective needle assembly.

Also or alternatively, in an embodiment of the invention, the connecting member comprises a thread for a thread connection or a guide structure for a twist connection.

Also or alternatively, in an aspect of the invention, the hub is adapted to be mounted on an injection device, the hub is adapted to be turned in a first direction until it reaches an end position. The hub comprises a first stop member adapted to cooperate with a corresponding second stop member on the injection device, at a position where the hub has reached the end position. The stop member of the hub is adapted to restrict the turning movement in the first direction.

That the connecting member, and thereby the needle assembly, is turnably connectable with a corresponding connecting member on an injection device, and that the cap portion can be turned from the initial to the broken mode provides a needle assembly, which enables a continuous process from mounting of the needle into breaking of the frangible zone, i.e., the same type of movement is used for mounting as for breaking. The needle assembly comprises a minimum a components as there is no need for an inner needle cap and an outer cap, which is seen in many prior art needle units for injection devices.

Also or alternatively, in an aspect of the invention, the cap portion is adapted to be operated between an end position mode, wherein the stop member of the hub restricts movement of the hub in the first direction, and the broken mode, wherein the cap portion and the hub can be separated.

Also or alternatively, in an aspect of the invention, the cannula cap portion, the hub, the frangible zone and the sealing member, comprises an outer surface surrounding an internal space of the needle assembly, and wherein the outer surface is adapted to preserve sterility of the internal space.

In some embodiments the needle cannula cannot be assembled with the hub, by insertion from the distal side of the hub, as the cap portion is a hindrance to this approach. In some embodiments the cap portion is not a hindrance to insertion from the distal side of the hub, but it may still be chosen to insert the needle from the proximal side. Therefore, also or alternatively, in an aspect of the invention, the central portion comprises a through-hole configured for receiving a needle cannula from the proximal side of the central portion.

Also or alternatively, in an embodiment of the invention, the needle cannula is conical or comprises a conical needle cannula portion. The central portion comprises a conical through-hole portion adapted to receive the conical needle cannula or the conical needle cannula portion. As the cannula and the through-hole is narrowing towards the distal end, the cannula and the through-hole are configured for a proximal insertion of the needle cannula into the hub, i.e., the needle cannula is inserted from the proximal side of the hub. Also or alternative, the conical through-hole portion is having a proximal end with a first diameter and a distal end with a second diameter, wherein the first diameter is larger than the second diameter. Also or alternatively, the conical needle cannula or conical needle portion is adapted to be pressure fitted into the through-hole.

Also or alternatively, in an aspect of the invention, the needle cannula comprises an outer surface provided with a first insertion stop, and wherein the central portion comprises a through-hole having a second insertion stop adapted to cooperate with the first insertion stop. Also or alternatively, the second insertion stop is adapted to restrict a movement in a distal direction of the needle cannula.

Also or alternatively, in an aspect of the invention, the central portion comprises a through-hole having a distal portion or a distal portion and a centrally placed local portion, adapted to fit closely around the needle cannula, and a proximal portion adapted to leave a space between the needle cannula and an inner surface of the through-hole, whereby the proximal portion is adapted to function as a glue reservoir. Also or alternatively, the needle assembly is configured for injection moulding and it is ensured, that the mould can be released from the needle assembly, i.e., by a proper configuration of the needle assembly, it is ensured that the mould, including the mould portion forming the through-hole, is not entrapped within the needle assembly during the moulding process.

Also or alternatively, an inner surface of the cap portion provides a third insertion stop, wherein the third insertion stop is adapted to cooperate with the distal end of the needle cannula.

Also or alternatively, the needle cannula comprises a sharp proximal tip in a proximal end of the needle and a sharp distal tip at the distal end of the needle.

Also or alternatively, the needle cannula is received in the central portion, and the needle comprises a distal portion, extending from a distal surface of the central portion, and a proximal portion extending from a proximal surface of the central portion. Also or alternatively, the skirt portion accommodates the proximal portion of the needle cannula, which ensures that the proximal portion is protected.

Also, the components within the internal space can be adapted to allow sterilization of the internal space. Also or alternatively, the components are permeable to a sterilizing agent, or wherein the components are provided with openings, they allow the passage of the sterilizing agent, wherein the sterilizing agent is a gas, a vapour or hot steam. The components can be the central portion, or for a hub comprising an outer and an inner portion, the component can be an inner skirt portion. Also or alternatively, a component can be the needle cannula, i.e., as the needle cannula is hollow the sterilizing gas can flow or diffuse through the cannula.

Also or alternatively, the cap portion can comprise gripping wings allowing a user to apply a torque to the cap portion.

In order to increase the free annular space between the needle cannula and an inner surface of the cap portion, the inner diameter of the cap portion can be increased. Therefore, in an aspect of the invention, an inner diameter of the cap portion equals an inner diameter of the through-hole 71, in order to enable a mould release. The needle cannula to be received in the through-hole comprises a diameter of a hub interfacing portion, which is substantially the same as the inner diameter of the through-hole 71, and a distal diameter of a distal protruding portion, which is smaller than the diameter of the hub interfacing portion. In this way it is ensured that the cannula can be fixed to the hub, and the adaptation allows a larger free annular space. Also or alternatively, the portion of the needle cannula which is smaller than the diameter of the hub interfacing portion may be position within a distal portion of the hub interfacing portion. In this way, the distal portion of the hub interfacing portion leaves a free annular space between the outer surface of the cannula and the inner surface of the through-hole. Such a free annular space can increase the bending flexibility with respect to radial forces on the distal portion of the needle.

Also or alternatively, in an aspect of the invention, the central portion of the hub comprises an insert opening, wherein the insert opening is adapted to receive and support a hub insert portion. The hub insert portion comprises a through-hole adapted to receive a needle cannula, wherein an inner diameter of the cap portion equals an inner diameter of the insert opening, in order to enable a mould release.

Also or alternatively, the insert opening comprises an inner surface comprising insert connecting means, wherein the hub insert portion comprises an outer surface comprising hub connecting means, wherein the insert connecting means and the hub connecting means are adapted to cooperate with each other, when the hub insert is inserted into the insert opening.

Also or alternatively, the insert connecting means and the hub connecting means comprises stop members providing a stop mechanism, when the insert is inserted into the hub. Also or alternatively, the hub insert portion comprises channels to allow sterilization of an internal space. Also or alternatively, the hub insert portion comprises a polymer or rubber that can be press-fitted into the hub. Also or alternatively, the hub insert portion comprises glue that can be hardened within the hub.

Also or alternatively, a distal portion of the insert portion comprises a free space or free annular space between an inner surface of the through-hole and an outer surface of the needle cannula. The free space or free annular space can constitute a glue tower, and can increase the flexibility of the needle cannula.

Also or alternatively, the central portion comprises a through-hole, wherein the hub is a one-piece unit, and wherein the needle cannula is a one-piece unit, and wherein the needle cannula interfaces directly with and inner surface of the through-hole. Such a design ensures a small number of complex components.

Also or alternatively, the hub further comprises an outer hub portion, and an inner hub portion, wherein the outer hub portion comprises an outer skirt portion, wherein the inner hub portion, is adapted to be received in the outer hub portion. The inner hub portion comprises an inner skirt portion and a central portion adapted to receive the needle cannula, and the inner skirt portion comprises a proximal surface adapted to attach to the sealing member, and an inner surface comprising a connecting member adapted to connect to an injection device. The two-part hub enables that a needle cannula can be inserted from a distal side of the hub, before the inner hub portion is received in the outer hub portion. Also or alternatively, the inner hub portion may comprise a through-hole with a distal portion comprising a free space or free annular space. The free space or free annular space can constitute a glue reservoir.

Also or alternatively, the needle assembly comprises a surface joint between the outer hub portion and the inner hub portion. The outer surface joint is adapted to be sealed by the sealing member. Also or alternatively, the needle assembly comprises a path between the outer hub portion and the inner hub portion. The path is adapted to be a tortuous path and thereby adapted to preserve a sterile condition of an internal space.

Also or alternatively, in an aspect the invention relates to a medical system comprising:
a needle assembly and
an injection device comprising a needle mount,
wherein the needle mount and the hub is adapted to be turnably connected, by turning the needle mount and the hub relative to each other,
wherein the hub comprises a first stop member and the needle mount comprises a second stop member, and
wherein the hub and the needle mount, during mounting of the hub on the injection device, is adapted to be turned relative to each other until they reach an end position, and
wherein the first stop member is adapted to cooperate with the corresponding second stop member, at a position where the hub and the needle mount has reached the end position, and
whereby the first stop member restricts the relative turning movement.

Also or alternatively, the cap portion is adapted to be operated between an end position mode, wherein the hub and the needle mount is positioned in the end position, and wherein the first stop member restricts relative movement of the hub and the needle mount, and a broken mode. In the broken mode, the hub and the needle mount are restricted in relative movement, and the cap portion and the needle mount are turned further relative to each other. The restriction between the first and the second stop member is adapted to sustain a torque, which is larger than a threshold torque required for breaking the frangible zone, and the cap portion and the hub can be separated from each other in an axial movement.

In one aspect, the cap and the hub can be turned in the same direction, wherein the direction can be a first direction, and wherein the first direction can be the turning direction of mounting the hub on the needle mount of the injection device.

Also or alternatively, in an aspect the invention relates to a method of mounting the needle assembly on an injection device comprising a needle mount, wherein the method comprises:
mounting the needle assembly on the needle mount by turning the cap portion and the needle mount, relative to each other until they reach an end position, whereby the hub and the needle mount are restricted in further rotation, and
further turning the cap portion and the needle mount relative to each other in a first direction, and thereby breaking the frangible zone.

Also or alternatively, a method of using the needle assembly comprises:
turning the cap portion and the hub relative to each other, and thereby breaking the frangible zone.

Also or alternatively, in an aspect the invention relates to a method of producing a needle assembly, wherein the method comprises:
producing a needle unit comprising a cannula cap portion, a needle hub and a frangible zone, wherein the frangible zone connects the needle hub and the cap portion, and wherein the needle hub is adapted to receive a needle cannula, and
inserting the needle cannula into the needle hub from a proximal side of the hub.

Also or alternatively, the method for producing a needle assembly comprises inserting the needle cannula until the needle cannula reaches a third insertion stop, and withdrawing the needle cannula until it has reached a desired position or has travelled a desired distance from the position, where it reached the third insertion stop.

All headings and sub-headings are used herein for convenience only and should not be constructed as limiting the invention in any way.

All examples, or exemplary language (e.g. such as) provided herein, is intended merely to better illuminate the invention and does not pose a limitation on the scope of the invention unless otherwise claimed. No language in the specification should be con-strued as indicating any non-claimed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### BRIEF DESCRIPTION OF DRAWINGS

In the following the invention will be further described with reference to the drawings, wherein

FIG. 1(a) shows a cross section of an embodiment of a needle assembly

FIG. 1(b) shows a perspective view of the needle assembly in FIG. 1(a)

FIG. 2(a) shows a schematic drawing of a conical needle cannula and needle hub

FIG. 2(b) shows a schematic drawing of a needle cannula and a needle hub having an insertion stop

FIG. 2(c) shows a schematic drawing of a needle cannula and a needle bub having a proximal glue reservoir

FIG. 3(a) shows a cross section of an embodiment of a needle assembly

FIG. 3(b) shows a perspective view of the needle assembly in FIG. 3(a)

FIG. 4 shows a needle cannula and needle hub

FIG. 5 shows a cross section of an embodiment of a needle assembly.

### DESCRIPTION

When in the following, terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only. In the following “distal” shall refer to the end closest to the patient, for a needle assembly in use for an injection. For example the distal tip of the needle is the tip inserted into the patient, when the needle is used for an injection. “Proximal” shall refer to the opposite of “distal”. For example the proximal tip of the needle is the tip pointing away from the patient, when the needle is used for an injection.

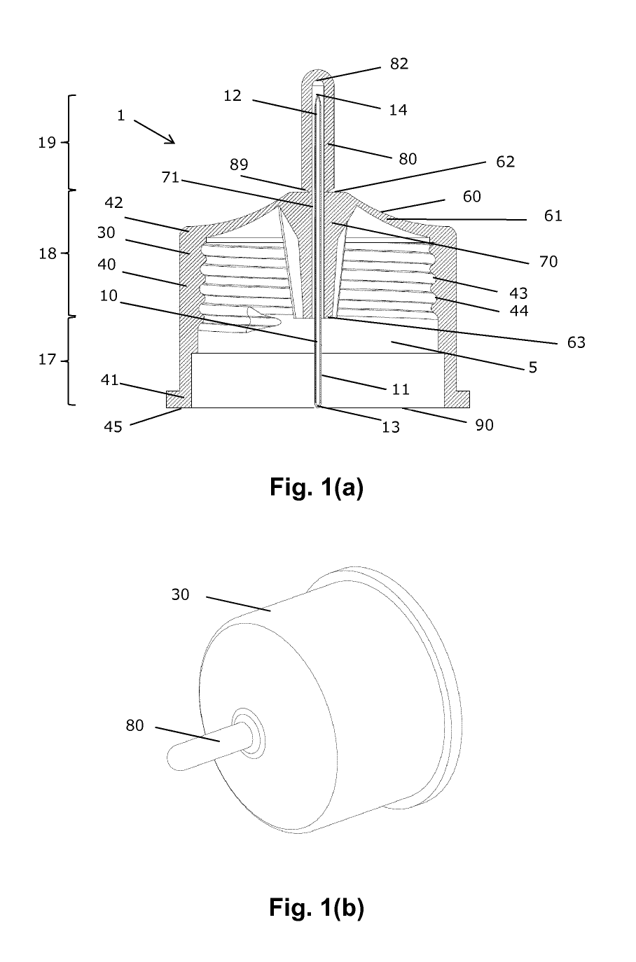

FIG. 1(a) and (b) show an embodiment of a needle assembly 1, and the needle assembly is to be used in connection with an injection device (not shown). The needle assembly comprises a needle cannula 10, a hub 30, a cap portion 80, and a frangible zone 89. The hub 30 comprises a skirt portion 40 and a central portion 60. The skirt portion 40 is having an internal surface 43 adapted for connection to an injection device, and the skirt portion 40 is having an open proximal end 41 adapted to be sealed off by a sealing member 90. The hub 30 is having a distal end 42 closed by the central portion 60, and the central portion 60 is adapted to receive the cannula 10. The internal surface 43 of the skirt portion 40, which is adapted for connection with an injection device, comprises a connecting member 44. The figure also shows a cap portion 80 covering a distal end of the cannula 12, and the frangible zone 89 connecting the cap portion with the central portion 70 of the hub. The cap portion 80 is adapted to be in an initial mode, wherein the frangible zone 89 connects the cannula cap portion 80 and the hub 30, or in a broken mode, wherein the frangible zone is broken, and the hub 30 and the cap portion 80 can be separated from each other. The cap portion 80 is manipulated from the initial mode to the broken mode by rotating the cap portion 80 relative to the hub 30. The connecting member 44 is adapted to be turnably connectable with a corresponding connecting member on an injection device, and the connecting member shown on FIG. 1 is a thread on the inner surface 43, adapted to turnably receive an outer thread of on a needle mount of an injection device.

The Connecting Member and the Frangible Zone

In some embodiments according to the invention, the connecting member comprises a thread for a thread connection or a guide structure for a twist connection. The guide structure can for example be a guide peg or a guide tab.

In some embodiments according to the invention, the hub 30 is adapted to be turned in a first direction for mounting on an injection device. The hub 30 comprises a first stop member adapted to cooperate with a corresponding second stop member on the injection device, at a position where the hub 30 has reached the end position. The stop member of the hub 30 is adapted to restrict the turning movement in the first direction, and the hub is, therefore, turned until it reaches the end position.

In some embodiments according to the invention, the cap portion 80 is adapted to be operated between an end position mode, wherein the stop member of the hub 30 restricts further movement of the hub 30 in the first direction, and the broken mode, wherein the cap portion 80 and the hub 30 can be separated, by pulling the cap portion 80 of in an axial or longitudinal movement. This feature can be accomplished by designing a stop member being able to resist the torque applied by the cap portion 80, and being able to restrict relative movement between the cap portion 80 and the hub 30, until the applied torque reaches a threshold and breaks the frangible zone 89.

Preservation of Sterility

In some embodiments, the cannula cap portion 80, the hub 30, the frangible zone 89 and the sealing member 90, comprises an outer surface surrounding an internal space 5 of the needle assembly, and wherein the outer surface is adapted to preserve sterility of the internal space, i.e., the outer surface prevents the entrance of bacterial organisms. In the embodiment in FIG. 1, the outer surface is a continuous surface.

In some embodiments, the skirt portion further comprises a proximal sealing surface 45 adapted to attach to the sealing member 90, and the sealing member 90 is adapted to attach to the sealing surface 45 for covering the proximal end of the hub. The attached sealing member 90 can be adapted to be peeled off.

Assembly of Needle Hub With Needle Cannula

In some embodiments according to the invention, the central portion 60 comprises a through-hole 71 configured for receiving a needle cannula from the proximal side of the central portion. FIG. 1 also shows a needle receiving portion 70, formed centrally in the central portion 60, and an annular portion 61 formed between the needle receiving portion and the skirt portion 40. The thickness of the needle receiving portion is thicker than the thickness of the annular portion, in order to increase the attachment or support surface between the needle cannula 10 and the hub 30. In some embodiments the cap portion 80, the frangible zone 89 and the hub 30 are moulded in one piece, and the through-hole 71 is not accessible from the distal side of the hub 30. The through-hole 71 is accessible from the proximal side, before the sealing member 90 is attached to the proximal surface.

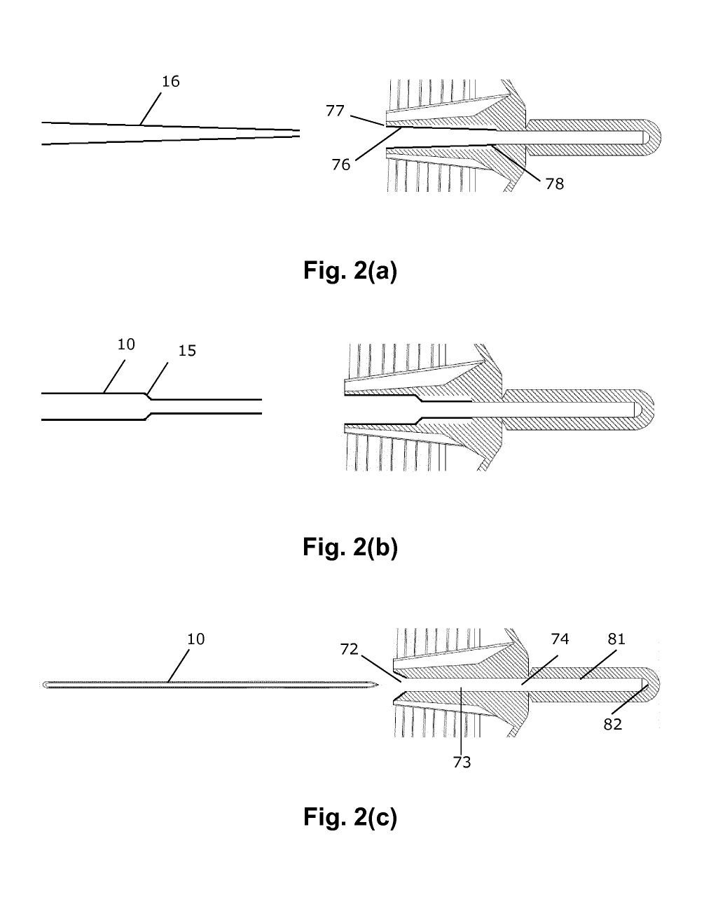

FIG. 2(a) shows an embodiments, where the needle cannula is a conical needle 16, and the central portion comprises a conical through-hole portion 76 adapted to receive the conical needle cannula. In some embodiments only a portion of the needle is conical, and this conical portion is then adapted to mate with the conical through-hole portion 76. The conical through-hole portion 76 may have the same extension as the entire through-hole.

In some embodiments, the conical through-hole portion 76 is having a proximal end 77 with a first diameter and a distal end 78 with a second diameter, wherein the first diameter is larger than the second diameter. In this way, the through-hole can be adapted to receive the needle from a proximal side. In some embodiments, the conical needle cannula 16 or a conical needle portion is adapted to be pressure fitted into the through-hole 76.

FIG. 2(b) shows an embodiment, where the needle cannula 10 comprises an outer surface provided with a first insertion stop 15, and wherein the central portion comprises a through-hole 71 having a second insertion stop 75 adapted to cooperate with the first insertion stop 15. In some embodiments, the second insertion stop 75 is adapted to restrict a movement in a distal direction of the needle cannula.

FIG. 2(c) shows an embodiment, where the central portion 60 comprises a through-hole 71 having a distal portion 74 and(/orXXX) a centrally placed local portion 73, adapted to fit closely around the needle cannula to avoid glue or to only allow a thin layer of glue between the needle cannula and the inner surface of the through-hole 71. The through-hole 71 also comprises a proximal portion 72 adapted to leave a space between the needle cannula and an inner surface of the through-hole, whereby the proximal portion is adapted to function as a glue reservoir 79. The proximal portion may be conical as shown on FIG. 2(c).

FIG. 2(c) also shows an embodiment, where an inner surface 81 of the cap portion provides a third insertion stop 82, wherein the third insertion stop 82 is adapted to cooperate with the distal end 12 or the distal tip 14 of the needle cannula 10.

The Needle

In some embodiments, the needle cannula is having a sharp proximal tip 13 in a proximal end 11 and a sharp distal tip 14 at the distal end 12, and wherein said needle cannula 10 is received in the central portion of the hub.

In some embodiments, as shown in FIG. 1(a), the needle cannula 10 is received in the central portion, and the needle comprises a distal protruding portion 19, extending from a distal surface 62 of the central portion 60, and a proximal protruding portion 17 extending from a proximal surface 63 of the central portion. FIG. 1(a) also shows a hub interfacing portion 18. In some embodiments and as shown in FIG. 1(a), the skirt portion 40 accommodates the proximal portion 17 of the needle cannula 10.

Sterilization of Needle Assembly

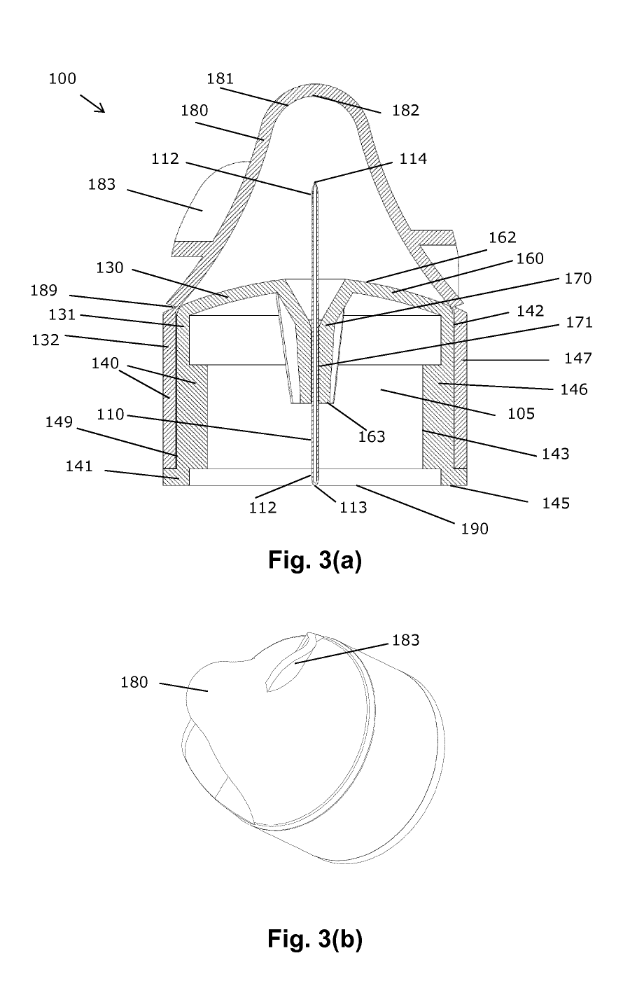

FIGS. 3(a) and 3(b) show another embodiment, of a needle assembly for an injection device. The needle assembly comprises a needle cannula 110, a hub 130 comprising a skirt portion 140 and a central portion 160. The skirt portion 140 is having an internal surface 143 adapted for connection to an injection device, or to a needle mount of an injection device. The skirt portion 140 is having an open proximal end 141 adapted to be sealed off by a sealing member 190. The hub 130 is having a distal end 142 closed by or connected to the central portion 160. The central portion 160 is adapted to receive the cannula 110, and the internal surface 143 is adapted for connection with an injection device comprises a connecting member. FIG. 3(a) also shows a cap portion 180 for covering the distal end 112 of the cannula 110 and a frangible zone 189. The cap portion 180 can be manipulated by a user from an initial mode to a broken mode. In the initial mode, the frangible zone 189 connects the cannula cap portion 180 and the hub 130. In the broken mode, the frangible zone 189 is broken, and the cannula cap portion 180 and the hub 130 can be separated from each other. The connecting member is adapted to be turnably connectable with a corresponding connecting member on an injection device, wherein the connecting member of the injection device may be positioned on or be a part of a needle mount.

In some embodiments, the sealing member 90, 190 is adapted to enable sterilization of the internal space 5, 105 by allowing the passage of a sterilizing agent, e.g., a sterilizing gas, sterilizing vapour or sterilizing hot steam.

In some embodiments, the components 10, 110, 146, 160 in the internal space 5, 105 are adapted to allow sterilization of the internal space 5, 105. The internal space 5, 105 can be adapted to such a purpose by ensuring that the sterilizing gas, after passage of the sealing member 90, 190, can circulate and enter all part of the internal space 5, 105.

In some embodiments, the components 10, 110, 146, 160 can be permeable to a sterilizing agent, or they are provided with openings, which allow the passage of the sterilizing agent. The sterilizing agent can be a gas or a vapour. In some embodiments the components can be the central portion 160 or an inner skirt portion 14. Alternatively or also, the components can be the needle cannula 10, 110.

Gripping Wings

In some embodiments, the cap portion further comprises gripping wings 183 allowing a user to apply a torque to the cap portion 180, i.e., the gripping wings eases the application of angular torque.

Inner Diameter of Cap Portion, Mould Release and Insert Portion

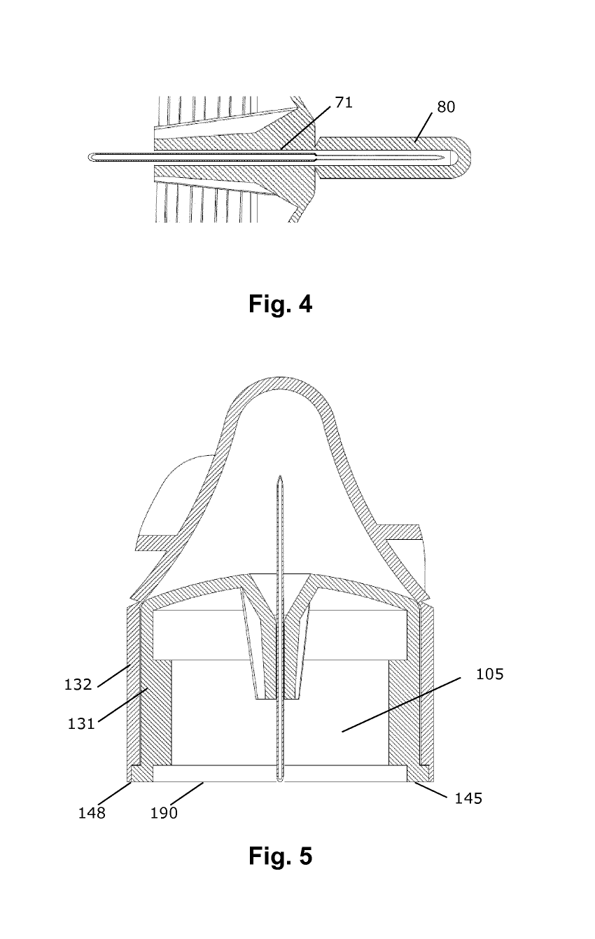

FIG. 4 shows a needle assembly, wherein an inner diameter of the cap portion 80 equals an inner diameter of the through-hole 71, in order to enable a mould release. The needle cannula 10 received in the through-hole 71 comprises a diameter of a hub interfacing portion 18, which is substantially the same as the inner diameter the through-hole 71 of the hub, and a distal diameter of a distal protruding portion 19, which is smaller than the diameter of the hub interfacing portion. In order to release an injection moulded needle assembly from a mould, the assembly should be designed in a way to ensure that the mould is not entrapped within the assembly.

In some embodiments, the central portion of the hub comprises an insert opening (not shown on figure). The insert opening is adapted to receive and support a hub insert portion (not shown on figure), wherein the hub insert portion comprises a through-hole adapted to receive a needle cannula, wherein an inner diameter of the cap portion equals an inner diameter of the insert opening, in order to enable a mould release. In some embodiments, the insert opening comprises an inner surface comprising insert connecting means, wherein the hub insert portion comprises an outer surface comprising hub connecting means, wherein the insert connecting means are adapted to cooperate with the hub connecting means, when the hub insert is inserted into the insert opening. In some embodiments the insert connecting means and the hub connecting means comprises stop members providing a stop mechanism, when the insert is inserted into the hub. In some embodiments, the hub insert portion comprises channels to allow sterilization of an internal space. In some embodiments, the hub insert portion comprises a polymer or rubber that can be press-fitted into the hub, and in some embodiments the hub insert portion comprises glue that can be hardened within the hub.

One Piece Unit

In some embodiments, as shown on FIG. 1, the hub 30 is a one-piece unit, and the needle cannula 10 is a one-piece unit. The needle cannula 10 received in the through-hole 71, interfaces directly with and inner surface of the through-hole 71. In embodiments, where the hub 30 is a one-piece unit and the cannula 10 is a one piece unit, it is not possible to attach the needle to a hub portion or an insert portion before the hub portion or insert portion is inserted into the rest of the needle assembly. In embodiments, where the hub 30 and cannula 10 are one-piece units, the needle cannula 10 is inserted into the hub 30 from the proximal side. The cannula is inserted until it reaches the desired position, and after it has reached the desired position, the needle cannula is attached to hub, e.g., by hardening a glue.

Two-Part Hub

In some embodiments, the hub 130 comprises an outer hub portion 132, and an inner hub portion 131. The outer hub portion 132 is adapted to be received in the inner hub portion 131, and the outer hub portion 132 comprises an outer skirt portion 147. The inner hub portion 131 comprises an inner skirt portion 146 and a central portion 160 adapted to receive the needle cannula 110, and the inner skirt portion 146 comprises a proximal surface 145 adapted to attach to the sealing member 190.

As shown on FIG. 5, in some embodiments, the needle assembly comprises a surface joint 148 between the outer hub portion 132 and the inner hub portion 131. The outer surface joint 148 is adapted to be sealed by the sealing member 190, i.e, the surface joint is positioned on a proximal surface of the hub, wherein the proximal surface is to be sealed off with a sealing member.

In some embodiments, as shown on FIG. 3(a), the needle assembly comprises a path 149 between the outer hub portion 132 and the inner hub portion 131. The path 149 is adapted to be a tortuous path, and it thereby preserves a sterile condition of the internal space 105 of the needle assembly.

Medical System

In some embodiments, a medical system comprises a needle assembly as described in the previous embodiments, and an injection device comprising a needle mount. The needle mount is adapted to be connected to the hub by turning the needle mount and the hub relative to each other. The hub comprises a first stop member and the needle mount comprises a second stop member. During mounting on the injection device, the hub and the needle mount is adapted to be turned relative to each other in a first direction until they reach an end position. At the end position, the first stop member is adapted to cooperate with the corresponding second stop member, and in this position the first stop member restricts the turning movement in the first direction.

In some embodiments, the cap portion is adapted to be operated between an end position mode, and a broken mode. In the end position mode, the hub is positioned in the end position, and the first stop member restricts movement of the hub in the first direction. In the broken mode, the hub is restricted in movement in the first direction, and the cap portion is turned further in the first direction. The restriction between the first and the second stop member is adapted to sustain a torque, which is larger than a threshold torque required for breaking the frangible zone.

Method of Mounting a Needle Assembly on an Injection Device

In some embodiments the invention also relates to a method of mounting a needle assembly on an injection device. In some embodiments the method of using the needle assembly comprises the step of mounting the needle assembly on a needle mount of an injection device by turning the cap portion and the needle mount relative to each other in a first direction until an end position. In the end position, the hub is restricted by the injection device in further rotation. The cap portion and the needle mount are turned beyond the end position, by continuing the relative movement in the first direction, and the frangible zone is thereby broken.

In some embodiments, the method comprises turning the cap portion and the hub relative to each other, and thereby breaking the frangible zone.

In some embodiments, the method comprises turning the cap portion and the hub in opposite directions, and thereby breaking the frangible zone.

Method of Producing a Needle Assembly

In some embodiments the invention relates to a method of producing a needle assembly. In some embodiments, the method of producing a needle assembly comprises the step of producing a needle unit comprising a cannula cap portion, a needle hub and a frangible zone. The frangible zone connects the needle hub and the cap portion, and the needle hub is adapted to receive a needle cannula. The needle assembly is assembled by inserting the needle cannula into the needle hub from a proximal side of the hub, as the distal side of the hub is covered by the cap portion and not available for needle insertion.

In some embodiments, a method for producing a needle assembly according to some of the embodiments, comprises inserting the needle cannula until the needle reaches a third insertion stop, withdrawing the needle cannula until it has reached a desired position or it has travelled a desired distance from the position, where it reached the third insertion stop. In the desired position the needle can be attached or fixed to the needle hub.

### List of Embodiments

1. A needle assembly (1, 100) for an injection device comprising:
a needle cannula (10, 110),
a hub (30, 130) comprising a skirt portion (40, 140) and a central portion (60, 160),
   wherein the skirt portion (40, 140) is having an internal surface (43, 143) adapted for connection to an injection device, wherein the skirt portion (40, 140) is having an open proximal end (41, 141) adapted to be sealed off by a sealing member (90, 190), wherein the hub (30, 130) is having a distal end (42, 142) closed by the central portion (60, 160), wherein the central portion (60, 160) is adapted to receive the cannula (10, 110), and
   wherein the internal surface (43, 143) of the skirt portion (40, 140) comprises a connecting member, wherein the connecting member is adapted to be turnably connectable with a corresponding connecting member on an injection device
a sealing member (90, 190),
a cap portion (80, 180) for covering the distal end (12, 112) of the cannula (10, 110),
a frangible zone (89, 189), and
wherein the cap portion (80, 180) can be turned from an initial mode,
a) wherein the frangible zone (89, 189) connects the cannula cap portion (80, 180) and the hub (30, 130),
   to a broken mode,
b) wherein the frangible zone (89, 189) is broken, and wherein the cannula cap portion (80, 180) and the hub (30, 130) can be separated from each other, and
a sterilizable mode, wherein the cannula cap portion (80, 180), the hub (30, 130), the frangible zone (89, 189) and the sealing member (90, 190), comprises an outer surface surrounding an internal space (5, 105) of the needle assembly, and wherein the outer surface is adapted to preserve sterility of the internal space, and
wherein the sealing member (90, 190) is permeable to a sterilizing agent, and is thereby adapted to enable sterilization of the internal space (5, 105).

2. A needle assembly according to embodiment 1, wherein the skirt portion (40, 140) further comprises a proximal sealing surface (45, 145) adapted to attach to the sealing member (90, 190), and the sealing member (190) is adapted to attach to the sealing surface (45, 145) for covering the proximal end (41, 141) of the hub (30, 130).

3. A needle assembly according to any of the previous embodiments, wherein the attached sealing member (90, 190) is adapted to be peeled off.

4. A needle assembly according to any of the previous embodiments, wherein the connecting member comprises a thread for a thread connection or a guide structure for a twist connection.

5. A needle assembly according to any of the previous embodiments, wherein the hub (30, 130), during mounting on an injection device, is adapted to be turned in a first direction until it reaches an end position, and
wherein the hub (30, 130) comprises a first stop member adapted to cooperate with a corresponding second stop member on the injection device, at a position where the hub has reached the end position, and
whereby the stop member of the hub (30, 130) is adapted to restrict the turning movement in the first direction.

6. A needle assembly according to embodiment 5, wherein the cap portion (80, 180) is adapted to be operated between
an end position mode, wherein the stop member of the hub (30, 130) restricts movement of the hub (30, 130) in the first direction,
and the broken mode, wherein the cap portion (80, 180) and the hub (30, 130) can be separated.

7. A needle assembly according to any of the previous embodiments, wherein the central portion (60, 160) comprises a through-hole (71, 171) configured for receiving the needle cannula (10, 110) from the proximal side of the central portion (60, 160).

8. A needle assembly according to any of the previous embodiments, wherein the needle cannula is conical (16) or comprises a conical needle cannula portion, and wherein the central portion (60, 160) comprises a conical through-hole portion adapted to receive the conical needle cannula (16) or the conical needle cannula portion.

9. A needle assembly according to embodiment 8, wherein the conical through-hole portion (76) is having a proximal end with a first diameter and a distal end with a second diameter, wherein the first diameter is larger than the second diameter.

10. A needle assembly according to any of embodiments 8-9, wherein the conical needle cannula (16) or conical needle cannula portion is adapted to be pressure fitted into the conical through-hole portion (76).

11. A needle assembly according to any of the previous, wherein the needle cannula (10, 110) comprises an outer surface provided with a first insertion stop (15), and wherein the central portion (60, 160) comprises a through-hole (71, 171) having a second insertion stop (75) adapted to cooperate with the first insertion stop (15).

12. A needle assembly according to embodiment 11, wherein the second insertion stop (75) is adapted to restrict a movement in a distal direction of the needle cannula (10, 110).

13. A needle assembly according to any of the previous embodiments, wherein the central portion (60, 160) comprises a through-hole (71, 171) having a distal portion (74) or a distal portion (74) and a centrally placed local portion (73) adapted to fit closely around the needle cannula (10, 110), and a proximal portion (72) adapted to leave a space between the needle cannula (10, 110) and an inner surface of the through-hole (71, 171), whereby the proximal portion (72) is adapted to function as a glue reservoir.

14. A needle assembly according to any of the previous embodiments, wherein an inner surface (81, 181) of the cap portion (80, 180) provides a third insertion stop (82, 182), wherein the third insertion stop (82, 182) is adapted to cooperate with the distal end (12, 112) of the needle cannula.

15. A needle assembly according to any of the previous embodiments, wherein the needle cannula (10, 110) comprises a sharp proximal tip (13, 113) in a proximal end (11, 111) and sharp distal tip (14, 114) at the distal end (12, 112), and wherein said needle cannula is received in the central portion (60, 160) of the hub (30, 130).

16. A needle assembly according to any of the previous embodiments, wherein the needle cannula (10, 110) is received in the central portion (60, 160), and wherein the needle comprises a distal protruding portion (19), extending from a distal surface (62, 162) of the central portion (60, 160), and a proximal protruding portion (17) extending from a proximal surface (63, 163) of the central portion (60, 160).

17. A needle assembly according to embodiment 16, wherein the skirt portion (40, 140) accommodates the proximal protruding portion (17) of the needle cannula (10, 110).

18. A needle assembly according to any of the previous embodiments, wherein components within the internal space (5, 105) is adapted to allow sterilization of the internal space.

19. A needle assembly according to any of the previous embodiments, wherein the components are permeable to a sterilizing agent, or wherein the components are provided with openings allowing the passage of the sterilizing agent, wherein the sterilizing agent is a gas or a vapor.

20. A needle assembly according to any of the embodiments 18-19, wherein the components can be the central portion (60, 160) or an inner skirt portion (146).

21. A needle assembly according to any of the embodiments 18-19, wherein the components can be the needle cannula (10, 110).

22. A needle assembly according to any of the previous embodiments, wherein the cap portion (80, 180) comprises gripping wings (183) allowing a user to apply a torque to the cap portion (80, 180).

23. A needle assembly according to any of the previous embodiments, wherein an inner diameter of the cap portion (80, 180) equals an inner diameter of the through-hole (71, 171), in order to enable a mould release, and wherein the needle cannula (10, 110) to be received in the through-hole (71, 171) comprises a diameter of a hub interfacing portion (18), which is substantially the same as the inner diameter of the through-hole (71, 171), and a distal diameter of a distal protruding portion (19), which is smaller than the diameter of the hub interfacing portion (18), and thereby allowing an increased space between the needle cannula and an inner surface of the cap portion.

24. A needle assembly according to any of the previous embodiments, wherein the central portion (60, 160) of the hub comprises an insert opening, wherein the insert opening is adapted to receive and support a hub insert portion, wherein the hub insert portion comprises a through-hole adapted to receive a needle cannula (10, 110), wherein an inner diameter of the cap portion (80, 180) equals an inner diameter of the insert opening, in order to enable a mould release.

25. A needle assembly according to embodiment 24, wherein the insert opening comprises an inner surface comprising insert connecting means, wherein the hub insert portion comprises an outer surface comprising hub connecting means, wherein the insert connecting means are adapted to cooperate with the hub connecting means, when the hub insert is inserted into the insert opening.

26. A needle assembly according to embodiment 25, wherein the insert connecting means and the hub connecting means comprises stop members providing a stop mechanism, when the insert is inserted into the hub.

27. A needle assembly according to any of embodiments 24-26, wherein the hub insert portion comprises channels to allow sterilization of an internal space.

28. A needle assembly according to any of embodiments 24-27, wherein the hub insert portion comprises a polymer or rubber that can be press-fitted into the hub.

29. A needle assembly according to any of embodiments 24-27, wherein the hub insert portion comprises glue that can be hardened within the hub.

30. A needle assembly according to any of embodiments 1-23, wherein the central portion (60) comprises a through-hole (71), wherein the hub (30) is a one-piece unit, and wherein the needle cannula is a one-piece unit, and wherein the needle cannula (10) interfaces directly with and inner surface of the through-hole (71).

31. A needle assembly according to any of embodiments 1-30, wherein the hub further comprises an outer hub portion (132), and an inner hub portion (131),
wherein the outer hub portion (132) comprises an outer skirt portion (147),
wherein the inner hub portion (131), is adapted to be received in the outer hub portion (132), the inner hub portion (131) comprises an inner skirt portion (146) and a central portion (160) adapted to receive the needle cannula (110), and the inner skirt portion (146) comprises a proximal surface (145) adapted to attach to the sealing member (190).

32. A needle assembly according to embodiment 32, further comprising a surface joint (148) between the outer hub portion (132) and the inner hub portion (131), and wherein the outer surface joint (148) is adapted to be sealed by the sealing member (190).

33. A needle assembly according to embodiment 32, further comprising a path (149) between the outer hub portion (132) and the inner hub portion (131), wherein the path (149) is adapted to be a tortuous path and thereby preserves a sterile condition of an internal space (105).

34. A medical system comprising:
a needle assembly (1, 100) according to any of the previous embodiments and
an injection device comprising a needle mount,
   wherein the needle mount and the hub (30, 130) is adapted to be turnably connected, by turning the needle mount and the hub (30, 130) relative to each other in a first direction,
   wherein the hub (30, 130) comprises a first stop member and the needle mount comprises a second stop member, and
   wherein the hub and the needle mount, during mounting of the hub on the injection device, is adapted to be turned relative to each other in a first direction until they reach an end position, and
   wherein the first stop member is adapted to cooperate with the corresponding second stop member, at a position where the hub and the needle mount has reached the end position, and
   whereby the first stop member restricts the relative turning movement in the first direction.

35. A medical system according to embodiment 35, wherein the cap portion (80, 180) is adapted to be operated between
an end position mode, wherein the hub (30, 130) and the needle mount is positioned in the end position, and wherein the first stop member restricts relative movement of the hub and the needle mount in the first direction,
and a broken mode, wherein the hub and the needle mount are restricted in relative movement in the first direction, and wherein the cap portion and the needle mount are turned further relative to each other in the first direction,
wherein the restriction between the first and the second stop member is adapted to sustain a torque, which is larger than a threshold torque required for breaking the frangible zone, and
wherein the cap portion and the hub can be separated from each other in an axial movement.

36. A method of mounting the needle assembly according to any of embodiments 1-33 on an injection device comprising a needle mount, wherein the method comprises:
mounting the needle assembly (1, 100) on the needle mount by turning the cap portion and the needle mount, relative to each other in a first direction until they reach an end position, whereby the hub (30, 130) and the needle mount are restricted in further rotation, and
further turning the cap portion (80, 180) and the needle mount relative to each other in the first direction, and thereby breaking the frangible zone.

37. A method of using the needle assembly according to any of embodiments 1-33, wherein the method comprises:
turning the cap portion (80, 180) and the hub (30, 130) relative to each other, and thereby breaking the frangible zone (89, 189).

38. A method of producing a needle assembly according to any of the embodiments 1-33, wherein the method comprises:
producing a needle unit comprising a cannula cap portion (80, 180), a needle hub (30, 130) and a frangible zone (89, 189), wherein the frangible zone connects the needle hub and the cap portion, and wherein the needle hub is adapted to receive a needle cannula, and
inserting the needle cannula (10, 110) into the needle hub from a proximal side of the hub.

39. A method according to embodiment 38 for producing a needle assembly according to embodiment 14 comprising,
inserting the needle cannula until the needle cannula reaches a third insertion stop (82, 182),
withdrawing the needle cannula until it has reached a desired position or travelled a desired distance from the position, where it reached the third insertion stop.

1. A needle assembly for an injection device comprising:
a needle cannula,
a hub comprising a skirt portion and a central portion,
wherein the skirt portion is having an internal surface adapted for connection to an injection device, wherein the skirt portion is having an open proximal end adapted to be sealed off by a sealing member, wherein the hub is having a distal end closed by the central portion, wherein the central portion is adapted to receive the cannula), and
wherein the internal surface of the skirt portion comprises a connecting member, wherein the connecting member is adapted to be turnably connectable with a corresponding connecting member on an injection device
a sealing member,
a cap portion for covering the distal end of the cannula,
a frangible zone, and
wherein the cap portion can be turned from an initial mode,
a) wherein the frangible zone connects the cannula cap portion and the hub,
to a broken mode,
b) wherein the frangible zone is broken, and wherein the cannula cap portion and the hub can be separated from each other, and
a sterilizable mode, wherein the cannula cap portion, the hub, the frangible zone and the sealing member, comprises an outer surface surrounding an internal space of the needle assembly, and wherein the outer surface is adapted to preserve sterility of the internal space, and
wherein the sealing member is permeable to a sterilizing agent, and thereby adapted to enable sterilization of the internal space.

2. A needle assembly according to claim 1, wherein the skirt portion further comprises a proximal sealing surface adapted to attach to the sealing member, and the sealing member is adapted to attach to the sealing surface for covering the proximal end of the hub.

3. A needle assembly according to claim 1, wherein the attached sealing member is adapted to be peeled off.

4. A needle assembly according to claim 1, wherein the connecting member comprises a thread for a thread connection or a guide structure for a twist connection.

5. A needle assembly according to claim 1, wherein the hub, during mounting on an injection device, is adapted to be turned in a first direction until it reaches an end position, and
wherein the hub comprises a first stop member adapted to cooperate with a corresponding second stop member on the injection device, at a position where the hub has reached the end position, and
whereby the stop member of the hub is adapted to restrict the turning movement in the first direction.

6. A needle assembly according to claim 5, wherein the cap portion is adapted to be operated between
an end position mode, wherein the stop member of the hub ) restricts movement of the hub in the first direction,
and the broken mode, wherein the cap portion and the hub can be separated.

7. A needle assembly according to claim 1, wherein the central portion comprises a through-hole configured for receiving the needle cannula from the proximal side of the central portion.

8. A needle assembly according to claim 1, wherein the needle cannula comprises an outer surface provided with a first insertion stop, and wherein the central portion comprises a through-hole having a second insertion stop adapted to cooperate with the first insertion stop.

9. A needle assembly according to claim 1, wherein the needle cannula comprises a sharp proximal tip in a proximal end and sharp distal tip at the distal end, and wherein said needle cannula is received in the central portion of the hub.

10. A needle assembly according to claim 1, wherein components within the internal space is adapted to allow sterilization of the internal space.

11. A needle assembly according to claim 10, wherein the components can be the central portion or an inner skirt portion.

12. A needle assembly according to claim 1, wherein the central portion of the hub comprises an insert opening, wherein the insert opening is adapted to receive and support a hub insert portion, wherein the hub insert portion comprises a through-hole adapted to receive a needle cannula, wherein an inner diameter of the cap portion equals an inner diameter of the insert opening, in order to enable a mould release.

13. A needle assembly according to claim 1, wherein the central portion comprises a through-hole, wherein the hub is a one-piece unit, and wherein the needle cannula is a one-piece unit, and wherein the needle cannula interfaces directly with and inner surface of the through-hole.

14. A needle assembly according to claim 1, wherein the hub further comprises an outer hub portion, and an inner hub portion,
wherein the outer hub portion comprises an outer skirt portion,
wherein the inner hub portion, is adapted to be received in the outer hub portion, the inner hub portion comprises an inner skirt portion and a central portion adapted to receive the needle cannula, and the inner skirt portion comprises a proximal surface adapted to attach to the sealing member.

15. A medical system according to claim 1 comprising:
a needle assembly according to any of the previous claims and
an injection device comprising a needle mount,
wherein the needle mount and the hub is adapted to be turnably connected, by turning the needle mount and the hub relative to each other in a first direction,
wherein the hub comprises a first stop member and the needle mount comprises a second stop member, and
wherein the hub and the needle mount, during mounting of the hub on the injection device, is adapted to be turned relative to each other in a first direction until they reach an end position, and
wherein the first stop member is adapted to cooperate with the corresponding second stop member, at a position where the hub and the needle mount has reached the end position, and
whereby the first stop member restricts the relative turning movement in the first direction.
